(19) United States
(12) Patent Application Publication
(10) Pub. No.: US 2023/0035942 A1
(43) Pub. Date: Feb. 2, 2023

(54) DELIVERY OF BIOLOGICS VIA MINERAL CRYSTALS FOR REGENERATIVE MEDICINE APPLICATIONS

(71) Applicant: Wisconsin Alumni Research Foundation, Madison, WI (US)

(72) Inventors: William L. Murphy, Waunakee, WI (US); Gianluca Fontana, Madison, WI (US); Andrew Khalil, Madison, WI (US); Jae-Sung Lee, Madison, WI (US)

(21) Appl. No.: 17/814,128

(22) Filed: Jul. 21, 2022

Related U.S. Application Data
(60) Provisional application No. 63/224,215, filed on Jul. 21, 2021.

(51) Int. Cl. A61K 9/50; C12N 15/88; A61K 48/00

(52) U.S. Cl. CPC A61K 9/501; C12N 15/88; A61K 48/0033

(57) ABSTRACT

Disclosed are compositions having mineral crystals and biological molecules for the delivery of the biological materials and methods for preparing mineral crystals having a biologic material.

### CROSS-REFERENCE TO RELATED APPLICATION(S)

This application claims priority to U.S. Application Ser. No. 63/224,215, filed Jul. 21, 2021, the disclosure of which is hereby incorporated by reference in its entirety.

### STATEMENT OF GOVERNMENT SUPPORT

This invention was made with government support under HL093282 awarded by the National Institutes of Health. The government has certain rights in the invention.

### BACKGROUND OF THE DISCLOSURE

The present disclosure relates generally to the delivery of biologics. More particularly, the present disclosure is directed to mineral crystals for the delivery of a biologic material and methods for preparing mineral crystals having a biologic material.

Regenerative medicine relies heavily on the delivery of biologics to elicit specific and adequate cell responses aiming at correcting imbalances between catabolism and anabolism. In particular, the delivery of biologics such as nucleic acids, proteins or peptides was shown to increase significantly the regenerative potential of target tissues. However, despite the great therapeutic potential of biologics their use in clinical applications is still somewhat limited.

Current issues are mostly associated with inadequate delivery strategies. Biologics have low stability and in some cases they can have low solubility in aqueous solutions, thereby affecting their bioavailability. In other words, higher doses of biologics are generally required to obtain desired effects, thus increasing the likelihood of complications. Accordingly, there exists a need for alternative compositions and methods for delivering biologics.

### BRIEF DESCRIPTION

In one aspect, the present disclosure relates to a composition comprising a mineral crystal and a biological molecule.

In another aspect, the present disclosure relates to a method for delivering a biological molecule to an individual in need thereof, the method comprising: administering a composition comprising a mineral crystal and a biological molecule to the individual.

In yet another aspect, the present disclosure relates to a method for transfecting a cell, the method comprising: contacting a cell with a polynucleotide-crystal complex; and culturing the cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIG. 1 is a scanning electron micrograph (SEM) image depicting mineral nanocrystals produced at high temperature.

FIG. 2 is a SEM image depicting mineral crystals produced at room temperature.

FIG. 3 is a SEM image depicting mineral nanocrystals produced at high temperature following a purification step to remove amorphous particles.

FIG. 4A is a bright field microscope image depicting Cy5-labelled mineral nanocrystals.

FIG. 4B is a fluorescent image depicting Cy5-labelled mineral nanocrystals shown in FIG. 4A.

FIG. 5A is a fluorescent image depicting Jurkat cells transfected using mineral nanocrystals to deliver complexed mRNA after Day 1. Scale bar, 50 μm.

FIG. 5B is an overlay of a fluorescent image and bright field image depicting Jurkat cells transfected using mineral nanocrystals to deliver complexed mRNA after Day 1. Scale bar, 50 μm.

FIG. 5C is a fluorescent image depicting Jurkat cells transfected using mineral nanocrystals to deliver complexed mRNA after Day 2. Scale bar, 50 μm.

FIG. 5D is an overlay of a fluorescent image and bright field image depicting Jurkat cells transfected using mineral nanocrystals to deliver complexed mRNA after Day 2. Scale bar, 50 μm.

FIG. 5E depicts transfection efficiency of each platform using Jurkat cells in suspension.

FIG. 5F depicts transfection efficiency of each platform using human bone marrow cells in suspension.

FIG. 5G is an electron micrograph depicting a mineral coated microparticle (MCM) contacting a human mesenchymal stem cell.

FIG. 5H is an electron micrograph depicting a fluoride-doped mineral coated microparticle (MCM) contacting a human dermal fibroblast.

FIG. 5I is an electron micrograph depicting mineral crystals.

FIG. 6A is a fluorescent image depicting control Jurkat cells incubated with mineral nanocrystals without complexed mRNA. Scale bar, 100 μm.

FIG. 6B is a fluorescent image depicting Jurkat cells incubated with complexed mRNA. Scale bar, 100 μm.

FIG. 6C is a fluorescent image depicting Jurkat cells incubated with complexed mRNA on mineral nanocrystals prepared at 150° C. Scale bar, 100 μm.

FIG. 6D is a fluorescent image depicting Jurkat cells incubated with mRNA on mineral nanocrystals prepared at 150° C. Scale bar, 100 μm.

FIG. 6E is a fluorescent image depicting Jurkat cells incubated with complexed mRNA in the presence of transduction buffer. Scale bar, 100 μm.

FIG. 6F is a fluorescent image depicting Jurkat cells incubated with complexed mRNA on mineral nanocrystals prepared at 150° C. in the presence of transduction buffer. Scale bar, 100 μm.

FIG. 7A is a fluorescent image depicting human bone marrow cells transfected with mRNA encoding green fluorescent protein on mineral nanocrystals. Scale bar, 50 μm.

FIG. 7B is a fluorescent image depicting human bone marrow cells transfected with mRNA encoding green fluorescent protein (without mineral nanocrystals). Scale bar, 50 μm.

FIG. 8A is a bright field microscope image depicting mesenchymal stem cells depicting internalized mineral nanocrystals localized in proximity to the nucleus. Scale bar, 50 μm.

FIG. 8B is a fluorescence image depicting mesenchymal stem cells depicting internalized mineral nanocrystals localized in proximity to the nucleus.

FIG. 9 is a graph depicting the presence of mineral nanocrystals and amorphous particles in the supernatant (bottom line) and pellet (top line) by X-ray diffraction (XRD) analysis.

FIG. 10 is a SEM image depicting mineral crystal formation at room temperature. Scale bar, 2 μm.

FIG. 11A is a low magnification SEM image depicting the synthesis of mineral crystals using complexed plasmid DNA (pDNA) as a nucleation point. Scale bar, 20 μm.

FIG. 11B is a high magnification SEM image depicting a synthesized mineral crystal using complexed plasmid DNA (pDNA) as a nucleation point. Scale bar, 10 μm.

FIG. 12A is a SEM image depicting a control mineral crystal formed in the absence of mRNA. Scale bar, 2 μm.

FIG. 12B is a SEM image depicting a mineral crystal formed using mRNA as a nucleation point. Scale bar, 2 μm.

FIG. 12C is a SEM image depicting a mineral crystal formed using complexed mRNA as a nucleation point. Scale bar, 2 μm.

FIG. 12D is XRD spectra depicting mineral crystals complexed with mRNA as a nucleation point (middle line), complexed mRNA as a nucleation point (bottom line), and control mineral crystals without mRNA (top line).

FIG. 13A is a bright field image depicting control mineral crystals for experiments incorporating Cy5-labelled mRNA. Scale bar, 50 μm.

FIG. 13B is a fluorescence image depicting mineral crystals synthesized using Cy4-labelled mRNA as a nucleation point. Scale bar, 50 μm.

FIG. 13C is a fluorescence image depicting mineral crystals synthesized using complexed Cy4-labelled mRNA as a nucleation point. Scale bar, 50 μm.

FIG. 13D is a high resolution SEM image depicting control mineral crystals for experiments incorporating Cy5-labelled mRNA. Scale bar, 2 μm.

FIG. 13E is a high resolution SEM image depicting mineral crystals synthesized using Cy4-labelled mRNA as a nucleation point. Scale bar, 2 μm.

FIG. 13F is a high resolution SEM image depicting mineral crystals synthesized using complexed Cy4-labelled mRNA as a nucleation point. Scale bar, 2 μm.

FIG. 14A-14C are bright field images of crystals synthesized in ethylene glycol at room temperature after 5 minutes (FIG. 14A), overnight (FIG. 14B), and following sonication (FIG. 14C).

FIG. 15 is a graph depicting transfection of human bone marrow using complexed mRNA encoding for G-luc (4 days).

FIGS. 16A & 16B are SEM images depicting bone marrow (BM) cell interaction with crystals (FIG. 16A) and Jurkat cell interaction with crystals (FIG. 16B).

FIGS. 17A & 17B depict a screening design for the transfection of Jurkat cells using crystals. FIG. 17A are graphs summarizing the effects on metabolic activity and transfection efficiency of variables such as: type of microparticles, ratio of complexing agent to mRNA, number of microparticles/well and ratio of nanoparticles to complexed mRNA. FIG. 17B lists the variables tested and orders them by their effect on the transfection efficiency.

FIGS. 18A & 18B depict transfection of Jurkat cells in 3-dimensional fibrin clot-mimic gels. FIG. 18A shows that firefly luciferase activity was high in crystals and MCM, whereas firefly luciferase activity was low in cells transfected using the FMCM platform. FIG. 18B shows that the metabolic activity of cells transfected using crystals, MCM and FMCM was equivalent.

FIGS. 19A-19O depict normalized alkaline phosphatase activity and mineralization during osteogenic differentiation of hMSCs as analyzed in Example 17.

FIGS. 20A & 20B depict 0.5× F-Cit MCM delivery of BMP-2 and BMP-7 mRNA as analyzed in Example 18.

FIGS. 21A-21K depict 0.5× F-Cit MCM mediated delivery of osteogenic biologics as analyzed in Example 19.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In one aspect, the present disclosure is directed to a composition comprising a mineral crystal and a biological molecule.

The mineral crystal is mostly consisted of calcium phosphate inorganic material including hydroxyapatite, tricalcium phosphate, octacalcium phosphate, monocalcium phosphate monohydrate, monocalcium phosphate anhydrous, dicalcium phosphate dihydrate, and dicalcium phosphate anhydrous. The mineral crystal can include dopants, such as halogen ions (e.g., fluoroide, chloride) and/or metal ions (e.g., aluminum, magnesium, strontium, manganese, iron, zinc, cobalt and silver). In one particularly suitable embodiment, the mineral crystal includes a transition metal, such as aluminum, magnesium, strontium, manganese, iron, zinc, cobalt, silver and combinations thereof.

The dissolution rate of the mineral crystal can be controlled by adjusting the concentrations of the mineral ions in the mineral precursor solution and/or by including dopants into the crystal structure.

The mineral crystal can range in diameter from about 50 nm to about 10 um, and including from about 50 nm to about 1 μm. In particularly preferred embodiments, the crystals are nanocrystals ranging in a diameter of from about 50 nm to less than 1 μm.

Suitable biological molecules include nucleic acids, proteins, peptides and combinations thereof. Suitable nucleic acids include DNA and RNA. The nucleic acid can be “naked” nucleic acids, complexed nucleic acids and combinations thereof. As used herein, “naked” nucleic acids (or “uncomplexed” nucleic acids) refers to nucleic acids that are incubated directly with the mineral crystal and not with any transfection reagent. As used herein, “complexed” nucleic acids refers to nucleic acids that are incubated with a transfection reagent such as a lipid-based transfection reagent to form a complex including the nucleic acid and the transfection reagent. By way of example only, complexed mRNA refers to a complex formed by mRNA and transfection reagent.

The biological molecule can be encapsulated in the mineral crystal, adsorbed to the surface of the mineral crystal, and combinations thereof.

The mineral crystal size can range from about 5 nm to about 20 μm, including a size ranging from about 5 nm to about 20 nm.

The morphology of the mineral crystals includes an amorphous crystal structure, a plate-like shape, a rhomboid shape, a flake-shape, and a rod shape.

In another aspect, the present disclosure is directed to a biologic delivery system. The biologic delivery system includes a mineral crystal and a biological molecule.

Suitable mineral crystals are described herein.

Suitable biological molecules are described herein.

The biological molecule can be encapsulated in the mineral crystal, adsorbed to the surface of the mineral crystal, and combinations thereof.

In another aspect, the present disclosure is directed to a method for delivering a biological molecule to an individual in need thereof. The method includes providing a composition including a mineral crystal and a biological molecule, wherein the biological molecule is encapsulated in the mineral crystal, adsorbed to the surface of the mineral crystal, and combinations thereof, and administering the composition to an individual in need thereof.

Suitable dosages of the compositions including the biological molecule encapsulated in the mineral crystal for use in the methods of the present disclosure will depend upon a number of factors including, for example, age and weight of an individual, severity of the disease to be treated, specific biological molecule to be used, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

Compositions of the present disclosure can further include pharmaceutically acceptable carriers. Pharmaceutically acceptable carriers may be, for example, excipients, vehicles, diluents, and combinations thereof. For example, where the compositions are to be administered orally, they may be formulated as tablets, capsules, granules, powders, or syrups; or for parenteral administration, they may be formulated as injections (intramuscular, subcutaneous, intramedullary, intrathecal, intraventricular, intravenous, intravitreal), drop infusion preparations, or suppositories. These compositions can be prepared by conventional means, and, if desired, the active compound (i.e., composition including a mineral crystal and biological molecule) may be mixed with any conventional additive, such as an excipient, a binder, a disintegrating agent, a lubricant, a corrigent, a solubilizing agent, a suspension aid, an emulsifying agent, a coating agent, or combinations thereof.

It should be understood that the pharmaceutical compositions of the present disclosure can further include additional known therapeutic agents, drugs, modifications of the synthetic compounds into prodrugs, and the like for alleviating, mediating, preventing, and treating the diseases, disorders, and conditions described herein.

The pharmaceutical compositions including the mineral crystal and biological molecule and/or pharmaceutical carriers used in the methods of the present disclosure can be administered to a subset of individuals in need. As used herein, an “individual in need” refers to an individual at risk for, having, and suspected of having a particular disease. Additionally, an “individual in need” is also used herein to refer to an individual at risk for, suspected of having or diagnosed by a medical professional as having the disease to be treated. As such, in some embodiments, the methods disclosed herein are directed to a subset of the general population such that, in these embodiments, not all of the general population may benefit from the methods. Based on the foregoing, because some of the method embodiments of the present disclosure are directed to specific subsets or subclasses of identified individuals (that is, the subset or subclass of individuals “in need” of assistance in addressing one or more specific conditions noted herein), not all individuals will fall within the subset or subclass of individuals as described herein. In particular, the individual in need is a human. The individual in need can also be, for example, a research animal such as, for example, a non-human primate, a mouse, a rat, a rabbit, a cow, a pig, and other types of research animals known to those skilled in the art.

In another aspect, the present disclosure is directed to a method for preparing a mineral crystal. A mineral crystal is synthesized by mixing two precursor solutions, one of which contains calcium ions and the other phosphate ions. Suitable mineral precursor solutions can include calcium chloride (CaCl₂), calcium nitrate (Ca(NO₃)₂), calcium hydroxide (Ca(OH)₂) for a calcium containing solution and disodium phosphate (Na₂HPO₄), monosodium phosphate (NaH₂PO₄), diammonium phosphate ((NH₄)₂HPO₄), dipotassium phosphate (K₂HPO₄), monopotassium phosphate (KH₂PO₄) for a phosphate containing solution. The method includes providing mineral precursor solution; mixing the mineral precursor solution at a temperature ranging from about 18° C. to about 200° C. for a time suitable for the formation of a mineral crystal.

Crystals can also be obtained by mixing supersaturated solutions of calcium and phosphate, respectively. These solutions can have H₂O or Ethylene Glycol (EG) as a solvent. Generally, crystals formed using EG as a solvent grow slower and in a more controlled manner relative to water solution. By way of example, in one particular embodiment, supersaturated solutions can be made by: preparing a 5M solution of Ca(OH)₂ in EG pre-heated to 100° C., preparing a 1M solution of (NH₄)₃PO₄ in EG pre-heated to 100° C. The solutions are allowed to cool down to 50° C. (thus increasing their saturation), and then filtered and mixed at a 1:1 ratio. Mineral crystals will start forming instantaneously and will grow over time. Incubations of 10 minutes at room temperature are sufficient to generate crystals. Their growth can be stopped by diluting the solution with EG and washing through repeated centrifugations.

In one embodiment, the method includes incubating the mineral precursor solution at a temperature ranging from about 80° C. to about 200° C. A particularly suitable temperature is about 150° C. In one embodiment, the method includes incubating the mixture at a temperature ranging from about 18° C. to about 25° C.

In one embodiment, the mineral precursor solution includes a biological molecule. Suitable biological molecules are described herein. Without being bound by theory, it is believed that the biological molecule provides a nucleation point for mineral formation.

The method can further include isolating the mineral crystals.

The method can further include contacting the mineral crystal with a solution including a biological material. Contacting the mineral crystal with a solution including the biological material results in adsorption of the biological material to the surface of the mineral crystal.

Mineral crystals can be formed without incorporating a biological material in the mineral crystal. Mineral crystals can be formed with a biological material incorporated in (i.e., encapsulated or embedded within) the mineral crystal. Mineral crystals can be formed without incorporating a biological material in the mineral crystal and with a biological material adsorbed to the surface of the mineral crystal. Mineral crystals can be formed with a biological material incorporated in (i.e., encapsulated or embedded within) the mineral crystal and with a biological material adsorbed to the surface of the mineral crystal. In embodiments of mineral crystals formed with a biological material incorporated in (i.e., encapsulated or embedded within) the mineral crystal and with a biological material adsorbed to the surface of the mineral crystal, the biological materials can be the same biological materials or different biological materials.

Various functions and advantages of these and other embodiments of the present disclosure will be more fully understood from the examples shown below. The examples are intended to illustrate the benefits of the present disclosure, but do not exemplify the full scope of the disclosure.

### EXAMPLES

### Example 1

In this Example, methods for preparing nanocrystals are described.

In the first method, nanocrystals were produced at 150° C. in ethylene glycol. Calcium nitrate (14 mM), precursor solution and disodium phosphate (0.3 M) precursor solution supplemented with 1.3 M sodium hydroxide were prepared in ethylene glycol. After two precursor solutions were heated to 150° C., phosphate precursor solution (1.36 mL) was added dropwise to the calcium precursor solution (50 mL) while stirring (mole ratio of Ca₂+ to PO₄³⁻=1.7:1). After the reaction at 150° C. for 5 minutes, the reaction was quenched by pouring the resulting mixture in the ice-cold acetone. The nanocrystals were washed with ethanol and deionized water, and collected by centrifugation. In the second method, nanocrystals were produced at room temperature using supersaturated solutions of calcium and phosphate. These solutions can have water or ethylene glycol as a solvent. Generally, crystals formed using ethylene glycol as a solvent grow slower and in a more controlled manner relative to water. Supersaturated solutions can be made by: preparing a 5 M solution of Ca(OH)₂ in ethylene glycol pre-heated to 100° C., preparing a 1 M solution of (NH₄)₃PO₄ in EG pre-heated to 100° C. The solutions were allowed to cool down to 50° C. (thus increasing their saturation), filtered and mixed at a 1:1 ratio. Mineral crystals started forming instantaneously and grew over time. Incubations of 10 minutes at room temperature were sufficient to generate nanocrystals. Their growth was stopped by diluting the solution with EG and washing through repeated centrifugations.

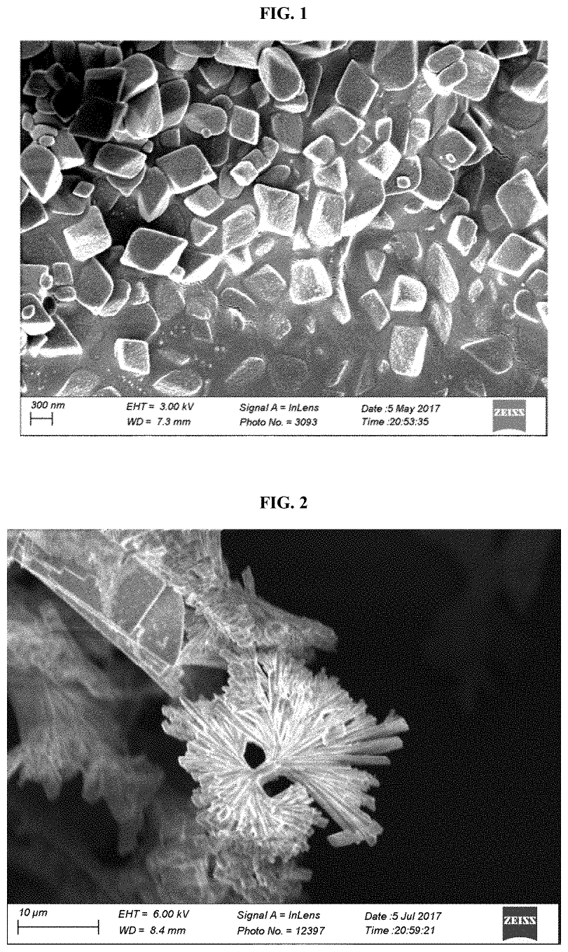

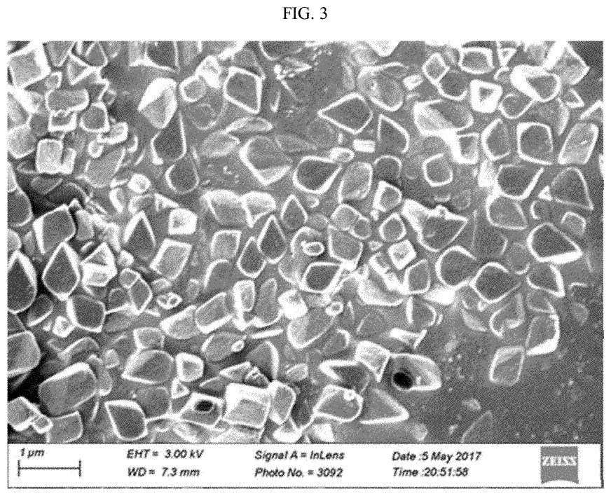

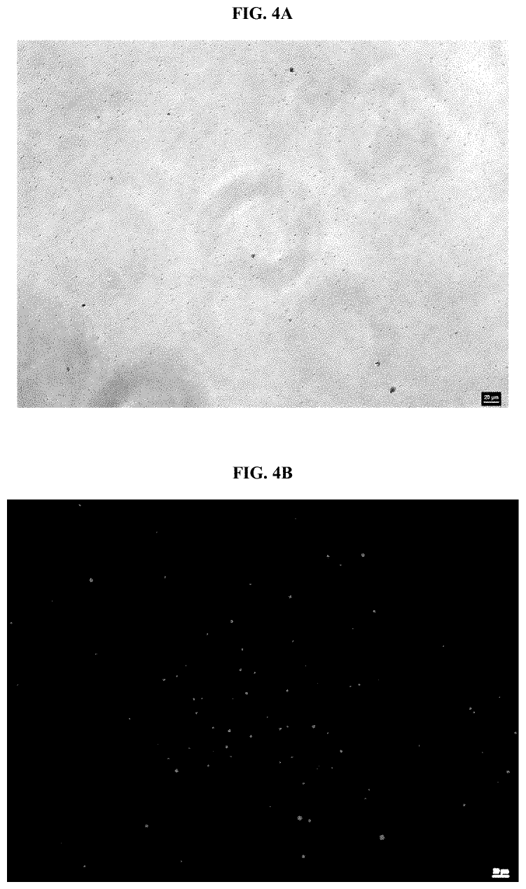

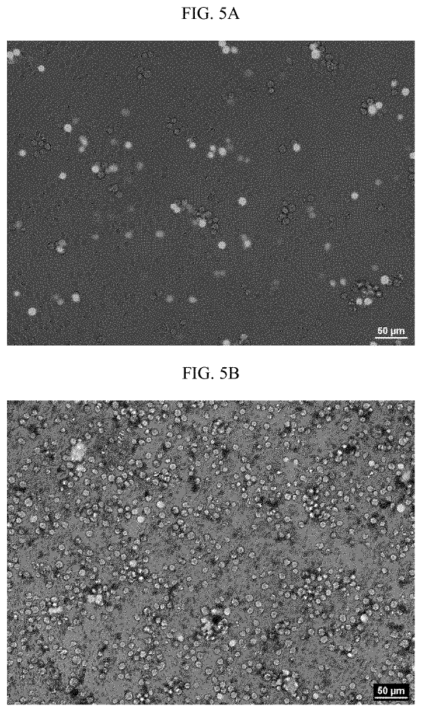

FIG. 1 depicts nanocrystals produced at 150° C. in ethylene glycol. The method produced a mixture of amorphous and crystalline products. FIG. 2 depicts nanocrystals produced at room temperature using supersaturated solutions. The method produced high crystallinity products. As shown in FIG. 3, smaller crystals (nanocrystals) formed at the higher temperature. Amorphous particles were also formed in the high temperature condition.

### Example 2

In this Example, incorporation of Cy5-labelled mRNA with nanocrystals prepared using the high temperature method was determined.

Nanocrystals were prepared using the high temperature method described in Example 1.

FIGS. 4A and 4B depict a bright field image (FIG. 4A) of nanocrystals and fluorescent image (FIG. 4B) showing Cy5 fluorescence.

### Example 3

In this Example, nanocrystals were used to transfect Jurkat cells (immortalized human T-cells) and human bone marrow cells with complexed mRNA.

mRNA encoding green fluorescent protein (GFP) was used to transfect Jurkat cells and human bone marrow cells. mRNA can be incubated with a lipidic transfecting agent and allowed to form nanocomplexes for 10 minutes at RT. Nanocrystals were added to the mRNA solution; for 1 μg of mRNA, were added 250 μg of nanocrystals. It should be understood that the reaction could be scaled up by maintaining the same ratios. mRNA complexes were allowed to interact with the nanocrystals for 1 hour at RT and then the unbound mRNA was washed off by centrifugation.

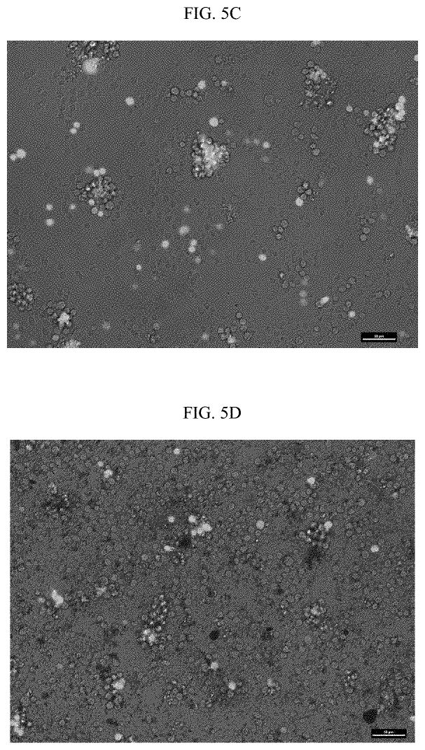

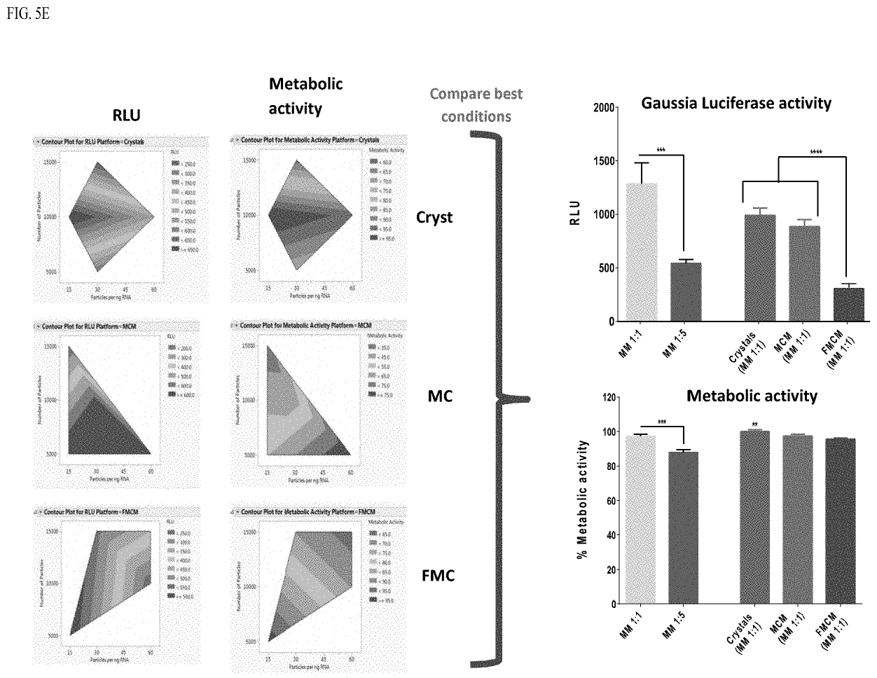

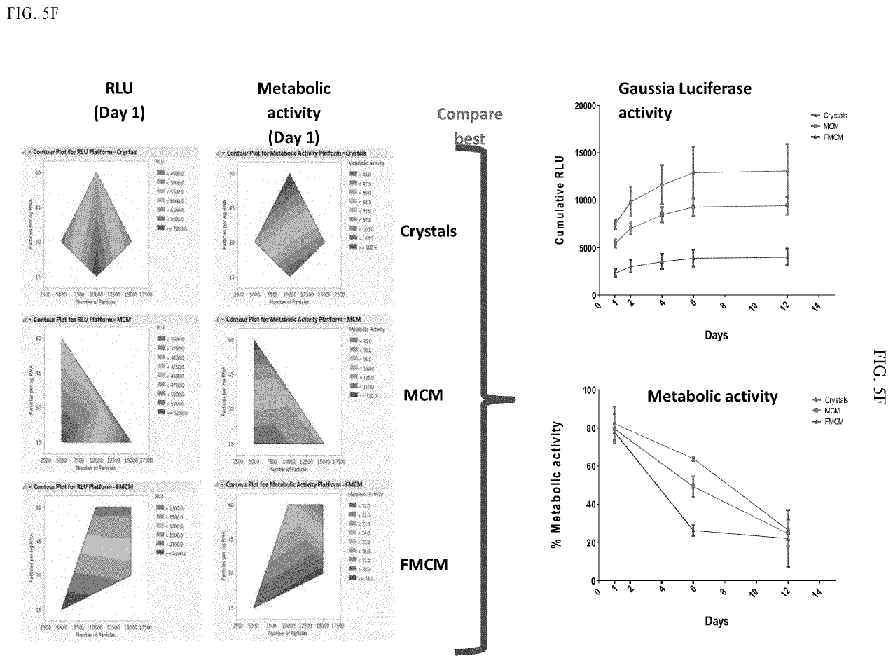

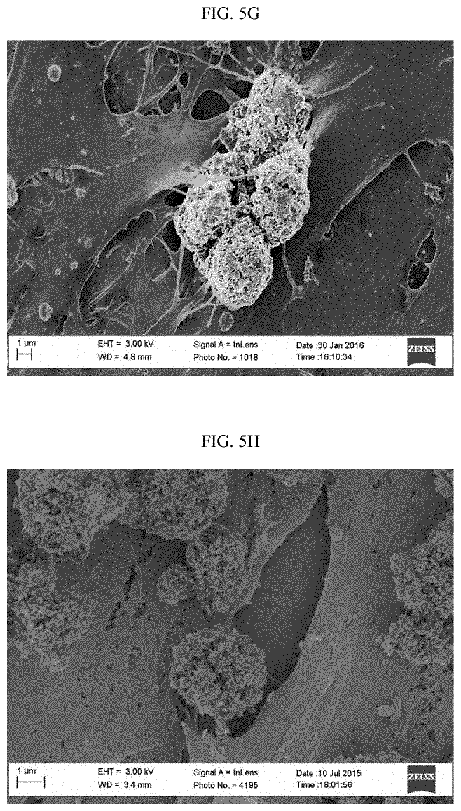

As depicted in FIG. 5A-5B, nanocrystals delivery of mRNA encoding GFP transfected both Jurkat cells (FIG. 5A) and human bone marrow cells (hBM; FIG. 5B). FIG. 5C is a fluorescent image depicting Jurkat cells transfected using mineral nanocrystals to deliver complexed mRNA after Day 2. FIG. 5D is an overlay of a fluorescent image and bright field image depicting Jurkat cells transfected using mineral nanocrystals to deliver complexed mRNA after Day 2. As illustrated in FIG. 5E, MCM resulted in similar transfection efficiency in Jurkat cells to crystals but with 4 times less RNA complexes. Crystals also resulted in a slightly higher transfection, a higher protective effect on the cells and a significantly higher metabolic activity despite delivering 4 times more RNA complexes. FMCM did not perform well with Jurkat cells. As illustrated in FIG. 5F, hBM showed similar response to mRNA transfection of Jurkat cells. Unexpectedly, the ratio 1:1 messenger max/mRNA showed the highest transfection as compared to the ratio 5:1. The metabolic activity of hBM cells decreased over time and the reporter mRNA was expressed for 6 days in vitro. Table 1 below summarizes the results depicted in FIG. 5E. Table 2 below summarizes the results depicted in FIG. 5F. FIGS. 5G-5I depict electron micrographs of MCM on a human mesenchymal stem cell (FIG. 5G), an electron micrograph of a fluoride-doped MCM on a human dermal fibroblast (FIG. 5H), and an electron micrograph of crystals (FIG. 5I) used for complexing with mRNA encoding GFP for transfection studies.

TABLE 1
Transfection efficiency of each platform in Jurkat cells.

| | | | Particles | | |
|---|---|---|---|---|---|
| | Ratio MM | Number of | per ng | | Metabolic |
| Platform | to mRNA | particles | mRNA | RLU | activity (%) |
| Crystals | 1 | 10000 | 15 | 719.75 ± 118.05 | 101.39 ± 11.63 |
| Crystals | 3 | 10000 | 60 | 502 ± 66.62 | 93.06 ± 8.88 |
| Crystals | 5 | 5000 | 30 | 255.5 ± 15.15 | 81.22 ± 9.21 |
| Crystals | 5 | 15000 | 30 | 192 ± 14.85 | 57.52 ± 8.22 |
| FMCM | 1 | 5000 | 15 | 598.75 ± 37.84 | 97.87 ± 3.92 |
| FMCM | 1 | 15000 | 30 | 517 ± 60 | 69.58 ± 5.57 |
| FMCM | 3 | 15000 | 60 | 400.25 ± 21.42 | 62.11 ± 9.85 |
| FMCM | 5 | 10000 | 60 | 214.5 ± 16.60 | 69.58 ± 8.26 |
| MCM | 1 | 5000 | 60 | 639.5 ± 81.63 | 85.17 ± 12.89 |
| MCM | 3 | 5000 | 15 | 728.5 ± 81.99 | 54.54 ± 22.15 |
| MCM | 3 | 10000 | 30 | 638.25 ± 28.16 | 41.09 ± 8.50 |
| MCM | 5 | 15000 | 15 | 126.25 ± 7.68 | 28.82 ± 13.07 |

TABLE 2
Transfection efficiency of each platform in hBM cells.

| | | | Particles | | |
|---|---|---|---|---|---|
| | Ratio MM | Number of | per ng | | Metabolic |
| Platform | to mRNA | particles | mRNA | RLU | activity (%) |
| Crystals | 3 | 10000 | 60 | 5905.25 ± 139.35 | 105.71 ± 2.52 |
| Crystals | 5 | 5000 | 30 | 4227.5 ± 141.89 | 97.15 ± 3.40 |
| Crystals | 5 | 15000 | 30 | 4393.25 ± 265.35 | 85.47 ± 2.76 |
| FMCM | 1 | 5000 | 15 | 2291.75 ± 394.11 | 77.95 ± 4.65 |
| FMCM | 1 | 15000 | 30 | 1839 ± 232.13 | 78.47 ± 3.82 |
| FMCM | 3 | 15000 | 60 | 1220.5 ± 187.65 | 70.43 ± 4.98 |
| FMCM | 5 | 10000 | 60 | 1214 ± 208.49 | 73.54 ± 2.59 |
| MCM | 1 | 5000 | 60 | 4142.25 ± 320.51 | 113.36 ± 6.60 |
| MCM | 3 | 5000 | 15 | 5412 ± 421.63 | 79.50 ± 7.71 |
| MCM | 3 | 10000 | 30 | 4619.25 ± 280.58 | 89.11 ± 3.32 |
| MCM | 5 | 15000 | 15 | 3129.75 ± 164.98 | 90.53 ± 2.42 |

### Example 4

In this Example, nanocrystals were used to transfect Jurkat cells with complexed mRNA.

Nanocrystals were synthesized using the first method described in Example 1.

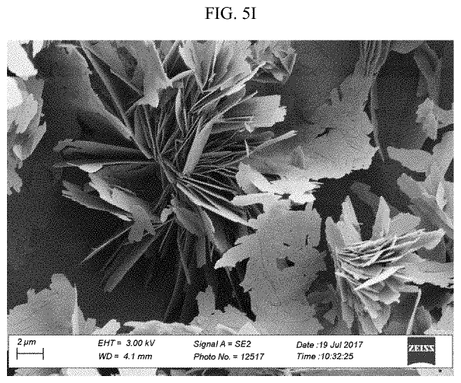

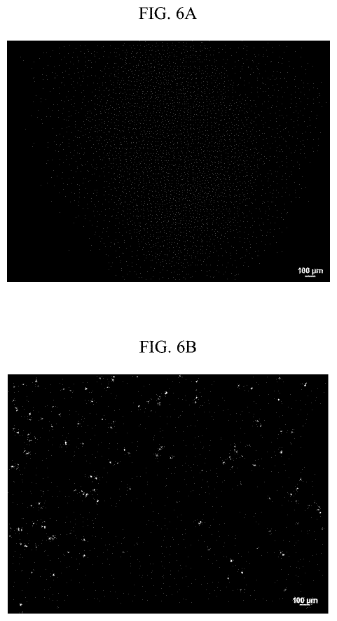

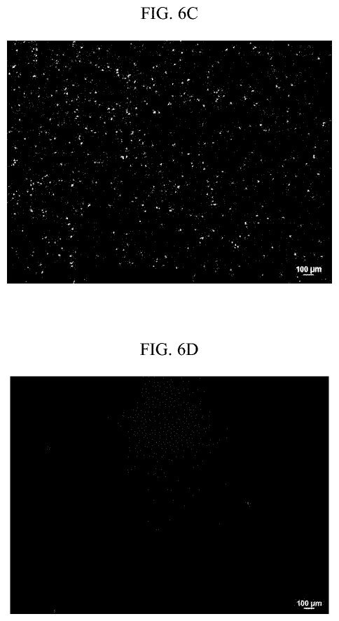

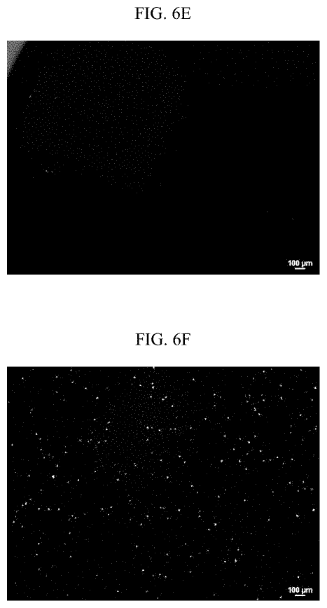

As depicted in FIGS. 6A-6F, Jurkat cells were transfected with complexed mRNA. Complexed mRNA was delivered in two ways: (i) adding complexed mRNA in the solution, and (ii) incorporating complexed mRNA on the mineral crystals. Loading mRNA complexes on nanocrystals appeared to increase transfection and maintained efficacy of complexes in the presence of transduction medium.

### Example 5

In this Example, nanocrystals were used to transfect human bone marrow cells with complexed mRNA.

Nanocrystals were prepared using the first method of Example 1 and complexed mRNA was prepared by the method described in Example 3.

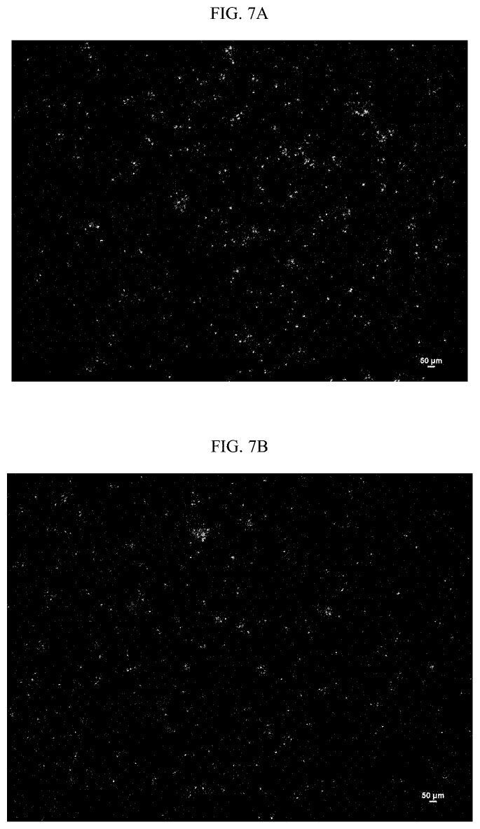

As depicted in FIGS. 7A and 7B, human bone marrow cells were transfected with complexed mRNA. Loading mRNA complexes on nanocrystals appeared to increase transfection and maintained efficacy of complexes in the presence of transduction medium.

### Example 6

In this Example, localization of nanocrystals in transfected mesenchymal stem cells was determined.

Nanocrystals were prepared using the first method of Example 1.

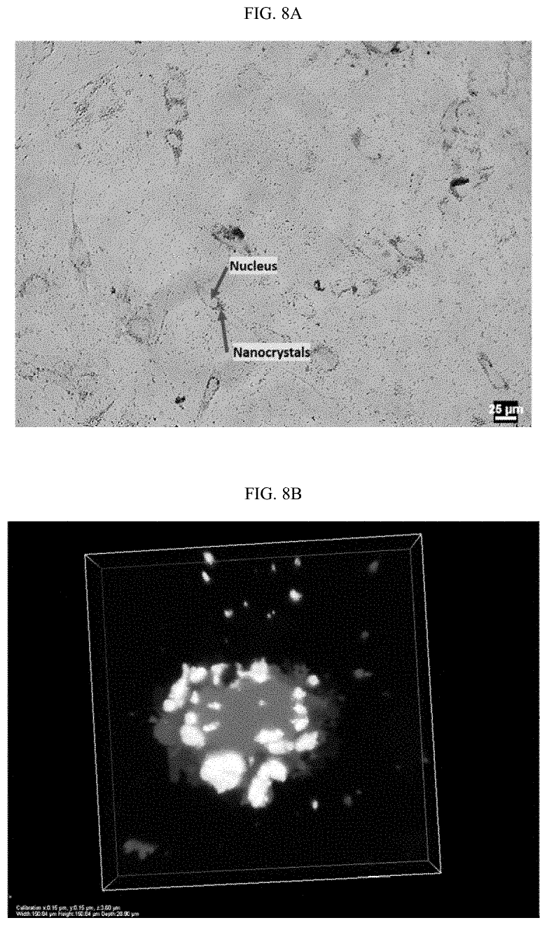

As depicted in FIGS. 8A and 8B, nanocrystals appeared to localize in proximity of the nucleus of the cell. Particularly, as shown in FIG. 8A, arrows indicate particle localization to the nucleus. In FIG. 8B, particles gathered around a single nucleus (shown in pink).

### Example 7

In this Example, precipitation of nanocrystals in solution was determined.

Nanocrystals were prepared using the first method of Example 1.

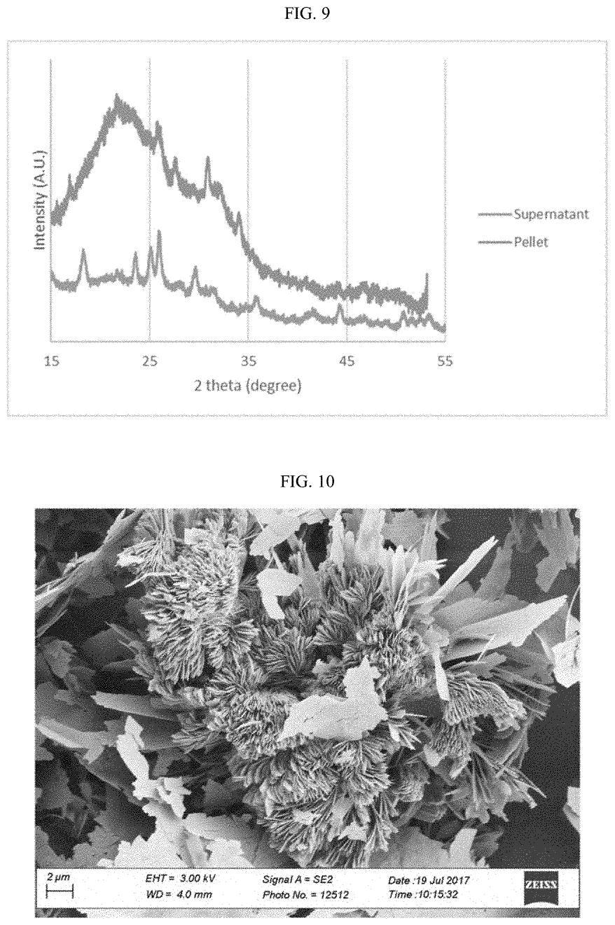

As depicted in FIG. 9, X-ray diffraction analysis demonstrated that amorphous particles precipitated more rapidly than nanocrystals.

### Example 8

In this Example, nanocrystals were prepared at room temperature.

Saturated solutions of calcium nitrate and disodium phosphate were mixed at room temperature as described in the second method of Example 1.

As depicted in FIG. 10, nanocrystals formed within milliseconds.

### Example 9

In this Example, complexed plasmid DNA (pDNA) was used as a nucleation point to prepare nanocrystals.

The nanocrystals were formed by mixing 1 ug of pDNA in 1 mL H₂0, 100 uL CaCl₂ 2 M, 1.1 mL Hepes and 220 uL of AlCl₃ 20 mM. AlCl₃ was used as a stabilizer to control the growth of the nanocrystals.

As depicted in FIGS. 11A (low resolution SEM) and 11B (high resolution SEM), pDNA provided a nucleation point for the formation of nanocrystals.

### Example 10

In this Example, complexed mRNA was used as a nucleation point to prepare nanocrystals.

A Ca/P solution was prepared using ethylene glycol (EG) as a solvent and by adding 0.164 g of calcium nitrate, 1.36 mL of Na₂HPO₄ (0.3 M) and 120 μL of NaOH 1.3 M to 50 mL of EG. 1 μg of mRNA was added to 1 mL of the Ca/P mix and crystals were grown for 10 minutes at room temperature. Naked mRNA or complexed mRNA served as a nucleation point, and as a result, it should be embedded within the newly formed crystals.

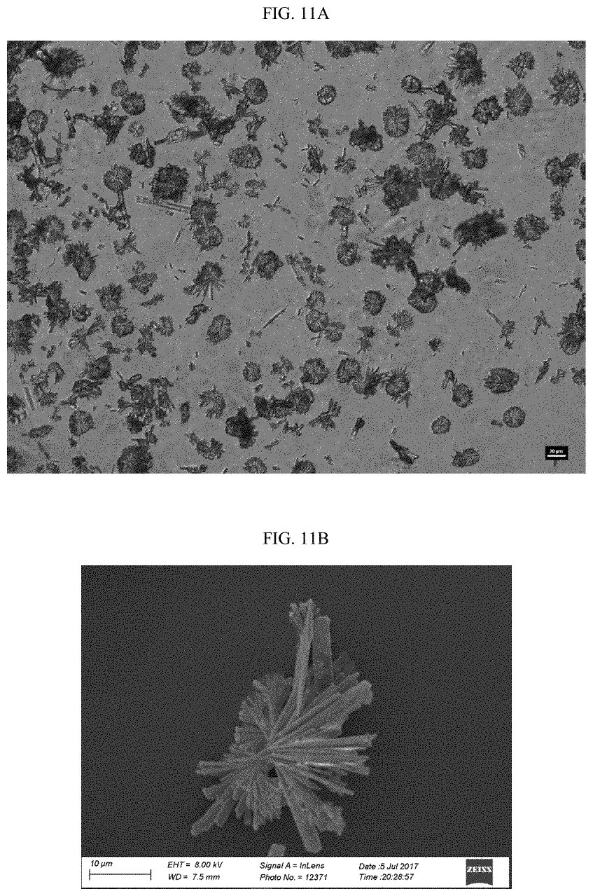

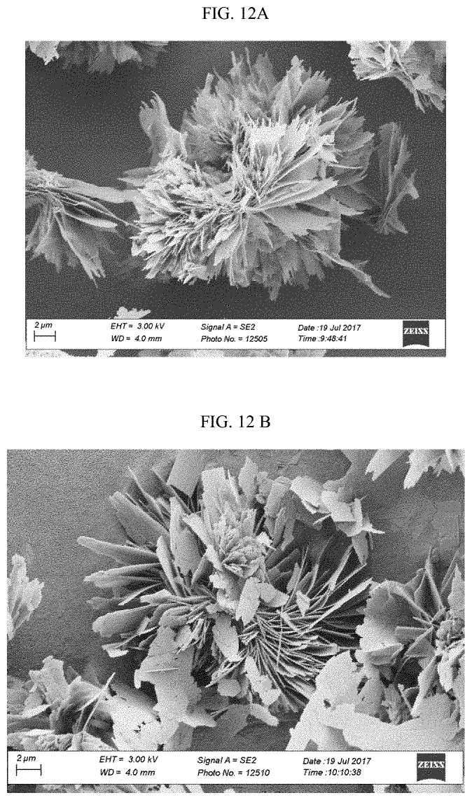

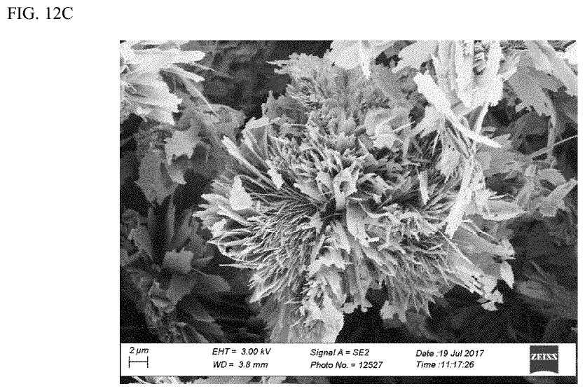

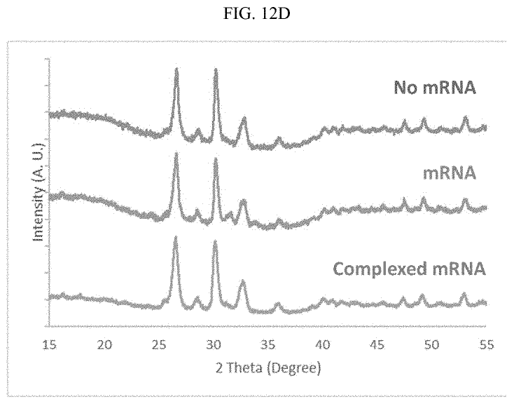

FIG. 12A is a SEM image showing control nanocrystals formed without the presence of mRNA. FIG. 12B is a SEM image showing nanocrystals formed using mRNA as a nucleation point. FIG. 12C is a SEM image showing nanocrystals formed using complexed mRNA as a nucleation point. FIG. 12D is a graph showing crystal structures are the same with or without mRNA and complexed mRNA as a nucleation point.

### Example 11

In this Example, Cy5-labelled mRNA was used as a nucleation point to prepare nanocrystals.

Complexed mRNA and nanoparticles were prepared and mixed as described in Example 10.

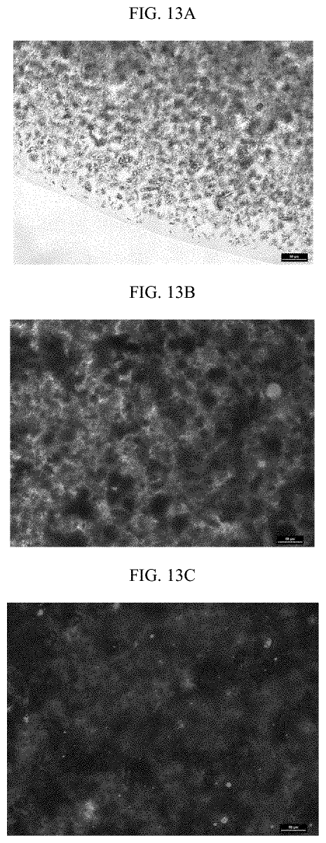

FIG. 13A is a bright field microscope image showing control nanocrystals formed without the presence of mRNA. FIG. 13B shows Cy5 fluorescence in nanocrystals formed using Cy5-labelled mRNA as a nucleation point. FIG. 13C shows Cy5 fluorescence in nanocrystals formed using complexed Cy5-labelled mRNA as a nucleation point. FIG. 13D is a SEM image of control nanocrystals (formed without mRNA). FIG. 13E is a SEM image of nanocrystals formed using Cy5-labelled mRNA. FIG. 13F is a SEM image of nanocrystals formed using complexed Cy5-labelled mRNA.

### Example 12

In this Example, nanocrystals were synthesized in ethylene glycol and reduced in size.

In particular, nanocrystals were made using the co-precipitation method described above. The sonication was performed by immersing a sonication probe in the solution containing the crystals and sonicated in cycles of 30 seconds. The sonicating probe tends to overheat, therefore after each cycle, the crystals solution was cooled in ice. 5 cycles were generally sufficient to reduce the size of the crystals.

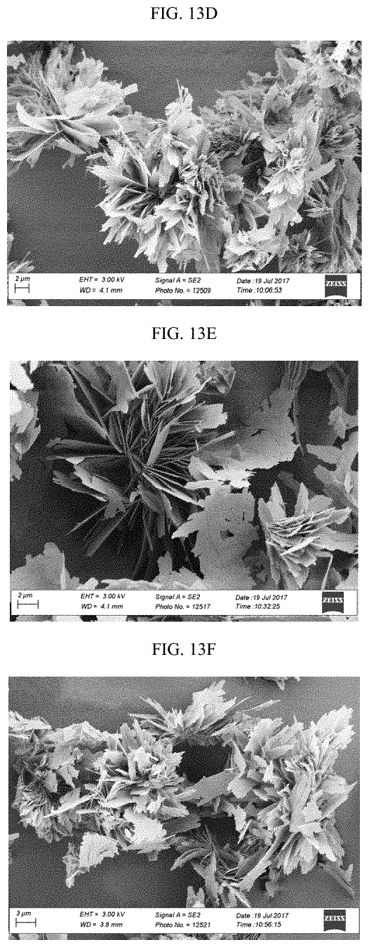

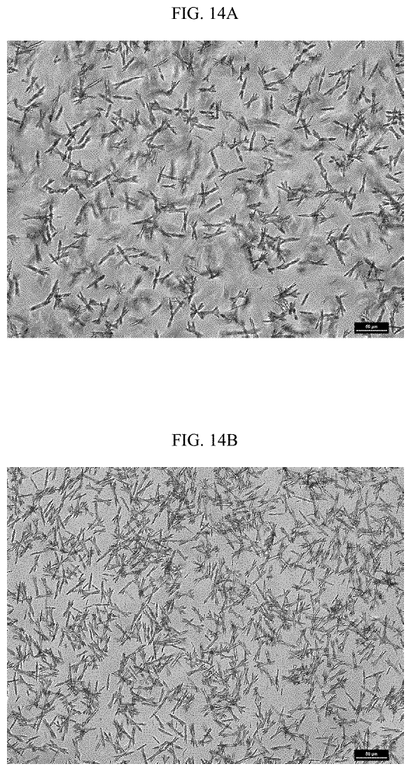

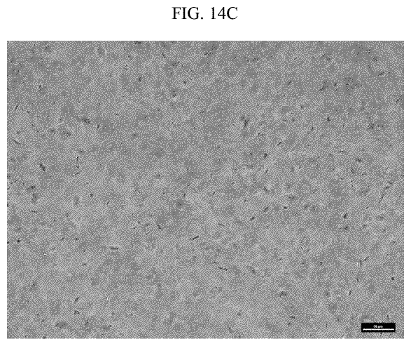

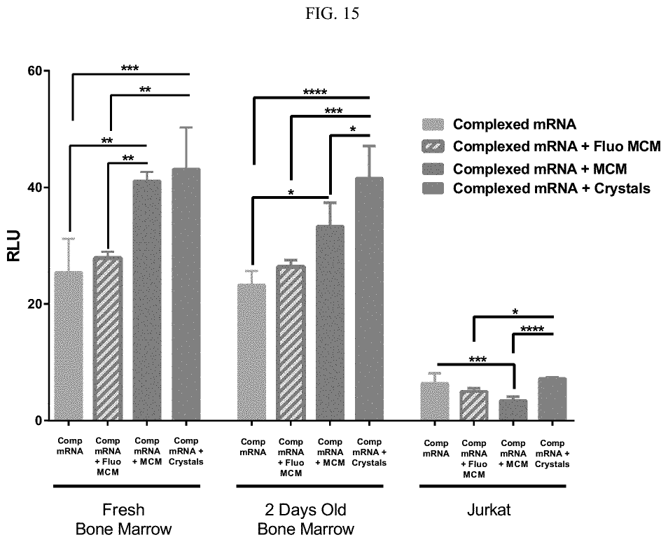

FIG. 14A depicts synthesized crystals after 5 minutes. FIG. 14B depicts synthesized crystals after overnight incubation. FIG. 14C depicts crystals after sonication.

### Example 13

In this Example, transfection of cells using nanocrystals complexed with mRNA was determined.

Experiments were performed in a 96-well plate seeded with 10⁵ cells (from fresh bone marrow, 2 day old bone marrow, and Jurkat cells) and 150 ng complexed mRNA per well. Crystals (about 20 μm in diameter) were synthesized in ethylene glycol and complexed with mRNA. Transfection using crystals was compared to mineralized beta-TCP (4.2 mM carbonate), mineralized beta-TCP fluoride-doped, and complexed mRNA.

### Example 14

In this Example, SEM was used to characterize bone marrow cells and Jurkat cells interacting with crystals.

Plastic coverslips were coated with a thin layer of fibrin. Bone marrow cells and Jurkat cells were mixed with crystals and then plated onto fibrin-coated coverslips. Cells were analyzed by scanning electron microscopy.

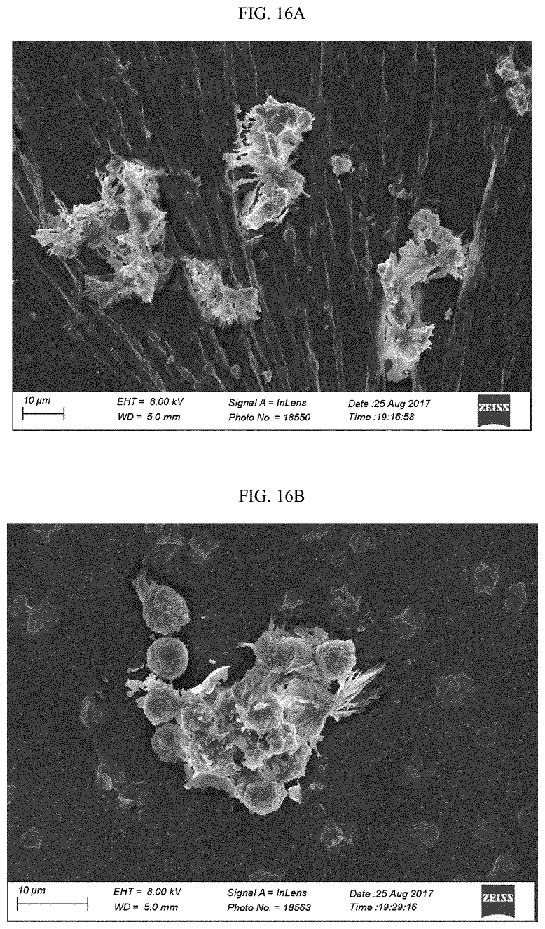

As shown in FIGS. 16A & 16B, both bone marrow cells (FIG. 16A) and Jurkat cells (FIG. 16B) interact with crystals. Bone marrow cells appear to wrap around the crystals (FIG. 16A).

### Example 15

In this Example, main effects screening design was used to assess conditions for transfecting Jurkat cells in vitro.

Crystals were compared to mineral coated microparticles (MCM) and fluoride-doped mineral coated microparticles (FMCM). Because each of these platforms have different masses, conditions were normalized by number of particles per well and particles per nanogram of mRNA. The output criteria used to establish the desirability of effects were metabolic activity and RLU.

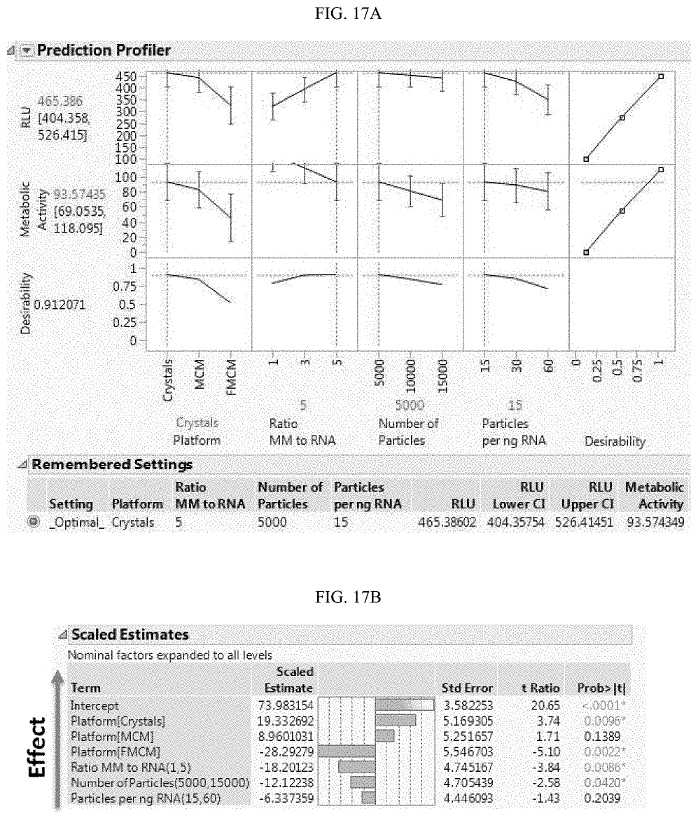

FIG. 17A shows the prediction profile for each platform. FIG. 17B summarizes the output criteria.

### Example 16

In this Example, mineral coated microparticles (MCM), fluoride-doped mineral coated microparticles (FMCM) and crystals were used to transfect Jurkat cells.

In particular, MCM, FMCM, and crystals were used for transfecting Jurkat cells in 3D fibrin clot-mimic gels and analyzed for firefly luciferase activity and metabolic activity.

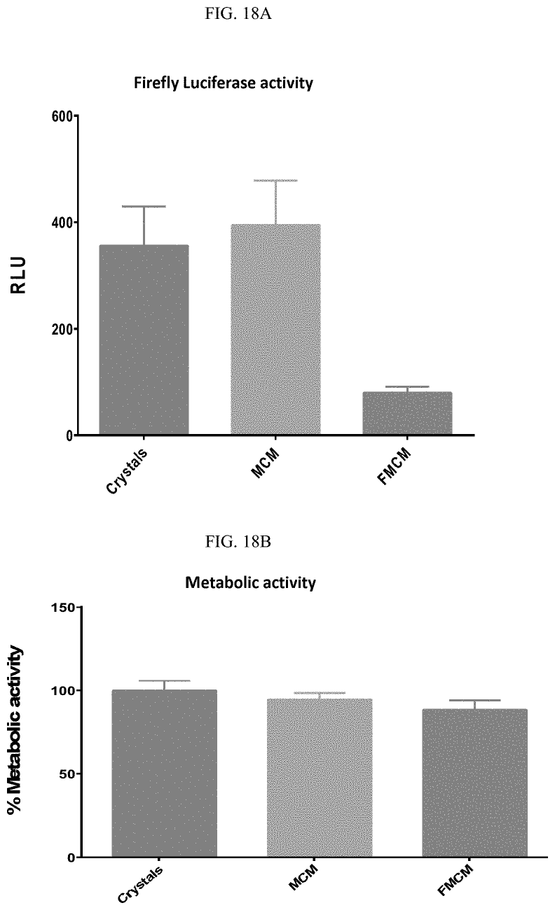

As depicted in FIG. 18A, firefly luciferase activity was high in crystals and MCM, whereas firefly luciferase activity was low in cells transfected using the FMCM platform. As depicted in FIG. 18B, the metabolic activity of cells transfected using crystals, MCM and FMCM was equivalent.

As disclosed herein, crystal-based delivery of biologics presents several potential advantages over existing platforms, including control over the dissolution rate of the mineral nanocrystals using different concentrations of mineral precursors. In addition, the biologics maintain their desired physical and chemical properties (i.e., activity) when encapsulated in mineral nanocrystals, which will eliminate the need to compensate for low-activity with higher concentrations and increased dosing. Moreover, crystallization allows for the delivery of biologics having low solubility into aqueous environments. Finally, the strong interaction between the nanocrystals and cells can serve to maximize the interaction between their cargo and the target cells.

### Example 17

In this Example, normalized alkaline phosphatase activity (ALP activity) and mineralization of mRNA for bone morphogenetic proteins during osteogenic differentiation of hMSCs were analyzed.

Tissue culture plates coated with 7.5 ug/mL of Rat Type 1 Collagen (BD) then cultured with human mesenchymal stromal Cells (hMSC; Lonza) were cultured in alpha-MEM (Thermofisher) with 1% Penicillin/Streptomycin and 10% FBS to 70% confluency. 0.5× F-Cit MCMs were prepared containing 0.5 fold the concentration of calcium:phosphate, 1 mM NaF and 5 mM Citric acid in the mSBF during preparation. hMSCs were treated with 0.5×-F-Cit MCMs+/−mRNA for BMP-2, BMP-7, BMP-2+BMP-7 or rhBMP-2 in osteogenic media or growth media. mRNA was complexed with Lipofectamine Messenger Max (Thermofisher) at 20 ug/mL for 5 minutes then bound to 0.5×-F-Cit MCMs at 120 ug 0.5×-F-Cit MCM:ug mRNA for 30 minutes. 0.5×-F-Cit MCMs were centrifuged at 2000 g for 30 seconds and then resuspended in media. Alkaline phosphatase (ALP; AnaSpec) and dsDNA (Thermofisher) content were determined at days 7, 14 and 21 of differentiation. Alizarin red staining was performed on PFA fixed cells at day 21.

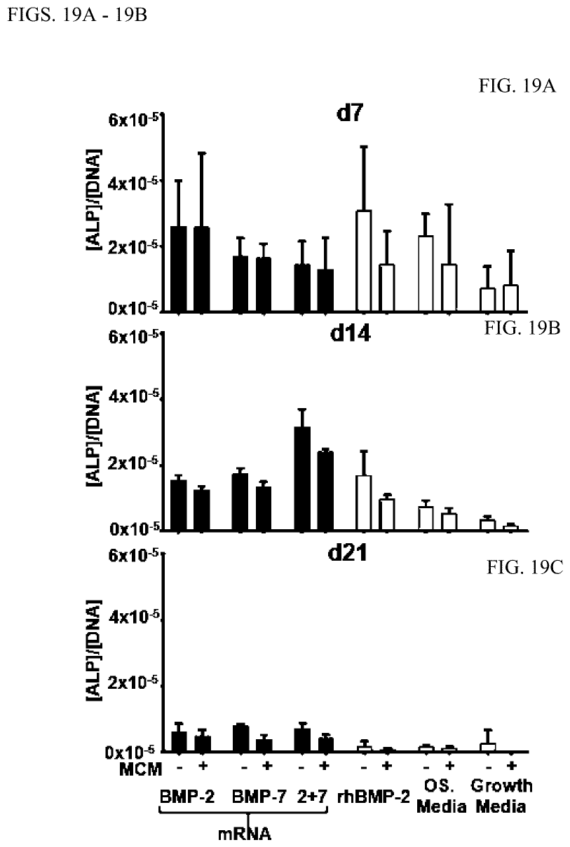

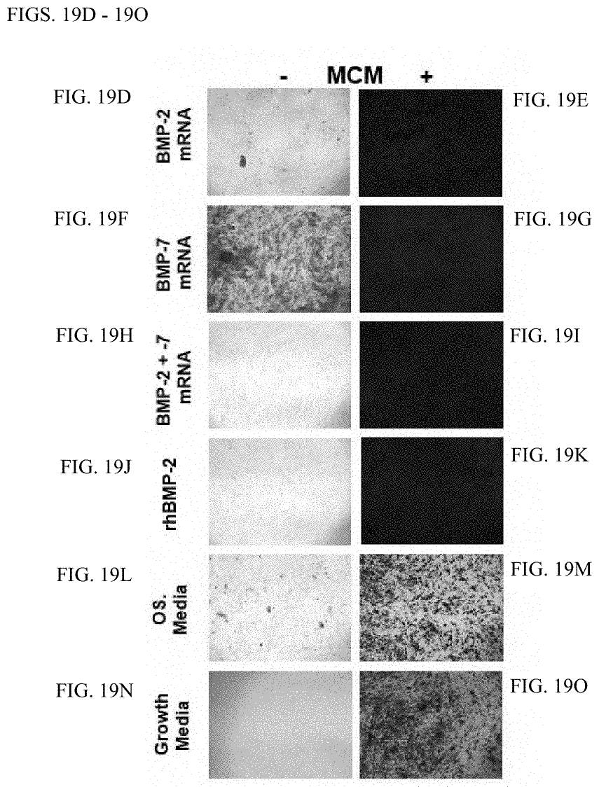

As shown in FIG. 19A, at day 7 post differentiation BMP-2 mRNA showed similar ALP activity normalized to ds DNA content to recombinant protein and more than BMP-7 or BMP2+BMP-7 mRNA. By day 14 (FIG. 19B), BMP-BMP-7 treated cells increased ALP expression the most and all declined by day 21 (FIG. 19C). MCM treatment tended to decrease ALP activity, but MCM groups demonstrated robust mineralization by Alizarin Red Staining at day 21 in all MCM treated groups including those without protein or mRNA (FIGS. 19D-19O). MCM treatment in growth media resulted in better mineralization at d21 than I osteogenic media (FIGS. 19D-19O).

### Example 18

In this Example, it is shown that delivery with 0.5× F-Cit MCM as prepared in Example 17 of BMP-2 and BMP-7 mRNA produces a heterodimeric BMP-2/-7 protein. Further, BMP-2 mRA can activated bone marrow cells.

50+50 ng of BMP-2+BMP-7 mRNA was delivered with or without 0.5×-FCit MCMs to hMSCs for 24 hours. Media was collected, MCMs were dissolved in 20 mM EDTA for 20 minutes at 37° C. and cell lysate were collected. BMP-2/-7 ELISA was performed using a BMP-7 capture and BMP-2 detection antibody. Recombinant BMP-2 and BMP-7 at 2 ng/mL were used as controls relative to MCMs alone (−/+) or untreated cells (−/−). Additionally, Rat tibial bone marrow was extracted and centrifuged using SepMate tubes (Stem Cell Technologies) and density gradient centrifugation with initial steps for 10 minutes at 1200 g and 2 washes with 2% FBS in PBS for 8 minutes each. Bone marrow aspirate concentrate (BMAC)was then cultured in IMDM Media (Gibco)+10% FBS, 1% Penicillin/Streptomycin. Cells were treated with 100 ng of BMP-2 mRNA+0.5× F-Cit MCMs or 25 ng rhBMP2 or MCMs alone.

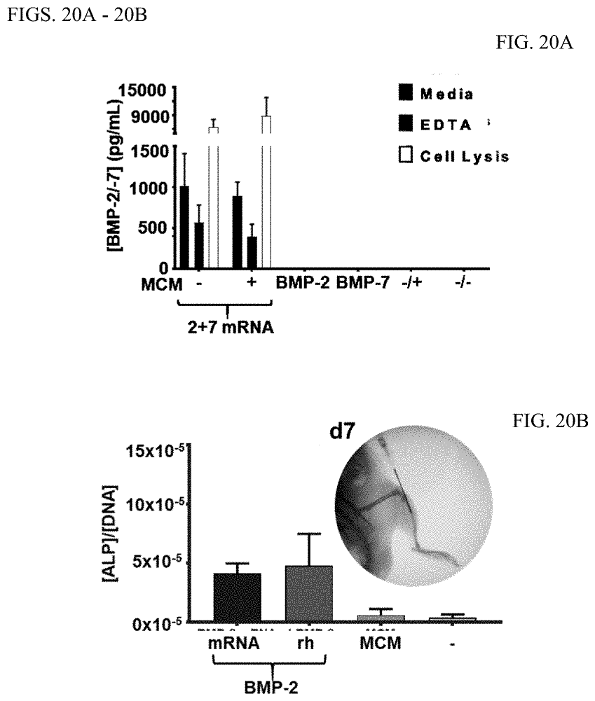

As shown in FIG. 20A, co-delivery of BMP-2 and BMP-7 mRNA produced a heterodimeric BMP-2/-7 protein. MCM mediated treatment increased the amount of protein produced in the lysate and most of the protein was in the lysate after 24 hours. FIG. 20B shows that treatment of rat BMAC with BMP-2 mRNA increased ALP activity similar to rHBMP-2 with minimal stimulation via MCMs alone.

### Example 19

In this Example, it is shown that 0.5× F-Cit MCM mediated delivery of osteogenic biologics promotes mineralization and ALP activation of rat bone marrow aspirates concentrate clots in vitro.

Rat bone marrow aspirate concentrate was harvested and cultured as described above. BMAC was treated with 0.5× F-Cit MCMs with mRNA for BMP-2 or BMP-2+BMP-7 versus recombinant protein and grown in IMDM media with osteogenic additives versus growth media (IMDM without osteogenic additives). Fibrin (0.2 mg/mL) and thrombin (2 u/mL) were added to the treated BMAC and incubated at 37° C. for 20 minutes to form a clot before media was added. BMAC-clots were cultured for 21 days then stained for Alizarin red or assayed for alkaline phosphatase activity and dsDNA content at days 7, 14, and 21.

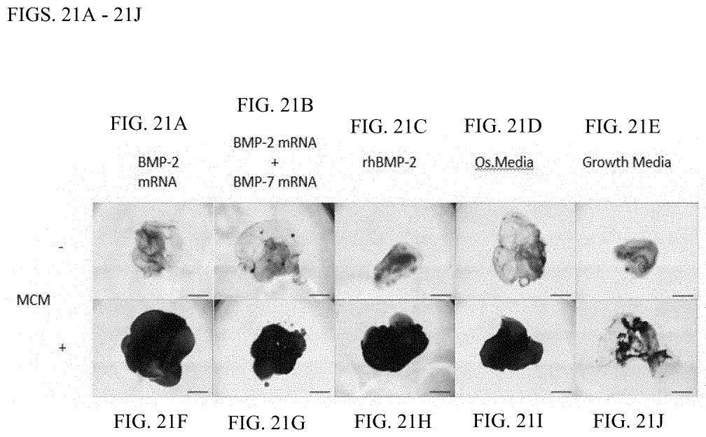

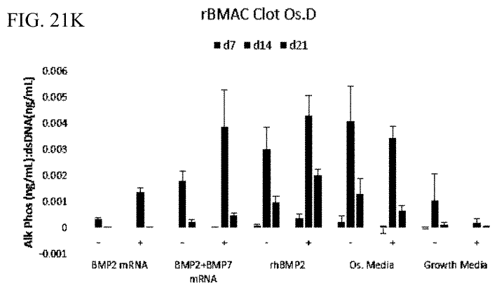

As shown in FIGS. 21A-21J, treatment of bone marrow aspirate concentrate clots with 0.5×-F-Cit MCMs improved osteogenic differentiation of bone marrow cells as assays by alizarin red staining at day 21. BMP-2+BMP-7 mRNA and rhBMP-2 resulted in slightly darker staining for mineralization than BMP-2 mRNA and 0.5× F-Cit MCMs alone. Treatment without MCMs or only in growth media resulted in minimal staining. In FIG. 21K, alkaline phosphatase activity normalized to dsDNA content showed minimal activation of BMAC in all groups at day 7 but robust activation by BMP-2+BMP-7 mRNA and rhBMP-2 at day 14 with declining activity by day 21. Treatment with 0.5×-FCit MCMs increased ALP activity over groups not including MCMs.

What is claimed is:

1. A composition comprising a mineral crystal and a biological molecule.

2. The composition of claim 1, wherein the mineral crystal comprises calcium nitrate (Ca(NO₃)₂) and disodium phosphate (Na₂HPO₄), and combinations thereof.

3. The composition of claim 1, wherein the mineral crystal comprises calcium chloride (CaCl₂).

4. The composition of claim 1, wherein the biological molecule comprises a nucleic acid, a protein, a peptide, and combinations thereof.

5. The composition of claim 1, wherein the biological molecule is encapsulated in the mineral crystal or adsorbed to the surface of the mineral crystal.

6. The composition of claim 1, wherein the biological molecule is encapsulated in the mineral crystal and adsorbed to the surface of the mineral crystal.

7. The composition of claim 6, wherein the biological molecule encapsulated in the mineral crystal and the biological molecule adsorbed to the surface of the mineral crystal are the same biological molecule.

8. The composition of claim 1, wherein the mineral crystal further comprises a transition metal, the transition metal selected from the group consisting of aluminum, magnesium, strontium, manganese, iron, zinc, cobalt, silver and combinations thereof.

9. The composition of claim 1, wherein the mineral crystal comprises a size ranging from about 5 nm to about 20 μm.

10. The composition of claim 9, wherein the mineral crystal is a nanocrystal comprising a size ranging from about 5 nm to about 20 nm.

11. The composition of claim 1, wherein the mineral crystal comprises one of an amorphous crystal structure, a plate-like nanostructure, a rhomboid shape, a flake-shape, and a rod shape.

12. The composition of claim 1, further comprising a pharmaceutically acceptable carrier.

13. A method for preparing a mineral crystal, the method comprising: providing a precursor solution comprising a calcium-containing supersaturated solution, a phosphate-containing supersaturated solution, and a solvent; heating the precursor solution to a temperature ranging from about 80° C. to about 200° C. for a time suitable for the formation of the mineral crystal.

14. The method of claim 13, wherein the calcium-containing supersaturated solution comprises at least one of calcium nitrate (Ca(NO₃)₂), calcium chloride (CaCl₂), and calcium hydroxide (Ca(OH)₂).

15. The method of claim 13, wherein the phosphate-containing supersaturated solution comprises at least one of disodium phosphate (Na₂HPO₄), monosodium phosphate (NaH₂PO₄), diammonium phosphate ((NH₄)₂HPO₄), dipotassium phosphate (K₂HPO₄), and monopotassium phosphate (KH₂PO₄).

16. The method of claim 13, wherein the solvent is selected from ethylene glycol and water.

17. The method of claim 13, wherein the time ranges from about 1 millisecond to 24 hours.

18. A method for delivering a biological molecule to an individual in need thereof, the method comprising: administering the composition of claim 1 to the individual.

19. The method of claim 18, wherein the biological molecule comprises a nucleic acid, a protein, a peptide, and combinations thereof.

20. The method of claim 18, wherein the administration comprises oral administration, injection, drop infusion preparations, suppositories and combinations thereof.

21. A method for transfecting a cell, the method comprising: contacting a cell with a polynucleotide-crystal complex; and culturing the cell.

22. The method of claim 21, wherein the crystal size ranges from about 5 nm to about 20 μm.
